(19) United States
(12) Patent Application Publication
(10) Pub. No.: US 2009/0317812 A1
(43) Pub. Date: Dec. 24, 2009

(54) METHOD OF DETECTING PREECLAMPSIA

(76) Inventors: Clifford L. Librach, Toronto (CA); Yie Shang-mian, Sichuan (CN); Li Liang-hong, Toronto (CA)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 12/306,749

(22) PCT Filed: Jun. 29, 2007
(86) PCT No.: PCT/CA2007/001168

§ 371 Date: May 27, 2009

Related U.S. Application Data
(60) Provisional application No. 60/806,307, filed on Jun. 30, 2006.

(51) Int. Cl. C12Q 1/68; C07H 21/04

(52) U.S. Cl. 435/6; 536/23.1

(57) ABSTRACT

The invention provides a method for detecting a propensity toward or risk of developing preeclampsia, comprising detecting in a female subject the presence of a single base pair point mutation, ΔA at position +1754, in the HLA-G mRNA 3′UTR. The mutation is associated with preeclampsic subjects, and with decreased RNA stability in vitro. The presence of this ΔA mutation may provide an explanation for lower levels of HLA-G expression seen in association with preeclampsia.

### CROSS REFERENCE TO RELATED APPLICATIONS

This application claims priority to and full benefit from U.S. Provisional Patent Application Ser. No. 60/806,307 filed Jun. 30, 2006, which is herein incorporated by reference.

### FIELD OF THE INVENTION

The present invention relates to a method for detecting preeclampsia or a propensity toward development of preeclampsia, and to detection kits.

### BACKGROUND OF THE INVENTION

Preeclampsia (PE) is a disease that affects approximately 5-10% of pregnant women and is one of major causes of maternal perinatal morbidity and mortality (1). Despite extensive study, its underlying etiology still remains elusive. However, it is generally agreed that preeclampsia is associated with shallow or absent placental cytotrophoblast invasion into the uterus (2). Many hypotheses have been put forward to explain the mechanisms for the development of preeclampsia. One hypothesis implicates a breakdown in the natural mechanism that protects the semi-allogeneic fetal allograft from rejection by the maternal immune system (3).

Human leukocyte antigen G (HLA-G) is a non-classical class I HLA molecule that is expressed by extravillous cytotrophoblast cells (4). It has been suggested that this protein may play a critical role in protection of cytotrophoblasts from maternal immune response, allowing these semi-allogeneic cells to invade the uterus unimpeded (5). Therefore, it has been proposed that the reduced HLA-G gene transcription (6-8) and translation (7-11) observed in women with preeclampsia may contribute to the pathogenesis of PE (5).

Since preeclampsia, according to epidemiological studies, has a strong familial component, it had been proposed that HLA-G may be an ideal candidate gene for mutations predisposing to PE (5). It has been reported that mutations of the HLA-G gene may be associated with reduced HLA-G gene transcription and translation, and thus may be involved in the pathogenesis of PE (12). However, opposite results have also been reported (13-14).

The steady-state levels of a particular mRNA species depend not only on its rate of synthesis but also on its rate of degradation. Adenylate/uridylate (AU)-rich element is a sequence consisting mostly of many uridines and some adenosines in the 3′-untranslated region (3′UTR) of mRNA and was first identified as a cis-acting degradation signal of the mRNAs of certain lymphokines, cytokines and proto-oncogenes (15, 16). Using RNA binding assays, several groups have identified proteins that interact with AU-rich elements and many of these proteins have been implicated in the regulation of mRNA stability (17-19). The 3′-UTR of HLA-G mRNA contains one AUUUA (SEQ ID NO: 1) motif and one AUUAUUUU (SEQ ID NO: 2) repeat.

### SUMMARY OF THE INVENTION

In one aspect described herein there is provided method of detecting a propensity toward or risk of developing preeclampsia comprising assessing a biological sample from a female subject for the presence of polymorphism ΔA at position +1754 in an HLA-G mRNA 3′UTR sequence, wherein the presence of polymorphism ΔA indicates a propensity toward preeclampsia or a risk of developing preeclampsia.

Additionally, there is described herein a method of detecting a propensity toward preeclampsia or a risk of developing preeclampsia comprising assessing a female subject for ΔA/ΔG or ΔA/ΔA genotype at position +1754 in HLA-G exon 8, containing HLA-G mRNA 3′UTR sequence.

Further, there is described herein the use of a sequence according to SEQ ID NO:3: 5′-TAAACTTTTTCATTTAAATA-3′, SEQ ID NO: 6, a modified sequence based thereon, or a sequence complementary thereto, for detection of a polymorphism ΔA at position +1754 in HLA-G mRNA 3′UTR in a biological sample from a female, indicative of a propensity toward or risk of developing preeclampsia. The modified sequence may comprise a substitution of one or more bases, a modification of one or more bases, a deletion of one or more bases, or a combination of these, that has no material effect on hybridization of the sequence to a sequence in the biological sample bearing the polymorphism ΔA.

Additionally, there is described herein a kit for detection of a propensity toward preeclampsia comprising: a probe for detecting polymorphism ΔA at position +1754 in HLA-G exon 8 in a biological sample from a female. The kit includes directions for use. The detection of polymorphism ΔA is indicative of a positive propensity or risk of developing preeclampsia.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached figures.

FIG. 1 shows a portion of the sequence of HLA-G mRNA 3′ untranslated region with poly A signal (SEQ ID NO: 4).

FIG. 2 illustrates HLA-G mRNA half-life evaluated by RT-PCR-Elisa.

FIG. 3 shows HLA-G mRNA expression determined by RNAase protection assay using Guaaauuuacuuuuucaaau (SEQ ID NO: 5).

FIG. 4 shows HLA-G mRNA expression determined by RNAase protection assay using Auaaauuuacuuuuucaaau (SEQ ID NO: 6).

FIG. 5 is a comparison of half-lives between HLA-G mRNA with mutation and control sequences.

### DETAILED DESCRIPTION

Generally, the present invention provides a method for detecting a propensity toward development of preeclampsia during a pregnancy. Based on this method, the risk that an individual when pregnant will go on to develop preeclampsia can be determined. This has the advantage of identifying an at-risk population prior to onset of the condition. Because of the serious nature of the condition, early detection or early risk assessment can have the result of reducing medical treatment required, and ultimately saving lives Detection of an individual who is at an early, pre-symptomatic stage of preeclampsia can be accomplished according to the invention, as well as confirmation of the condition in individuals presenting with symptoms. A kit for use in making such determinations is also described.

A method of detecting a propensity toward preeclampsia or risk of preeclampsia is described. The method comprises assessment of a biological sample from a female subject for the presence of polymorphism ΔA at position +1754 in an HLA-G mRNA 3′UTR sequence. The presence of polymorphism ΔA indicates a propensity toward preeclampsia or an increased risk for this condition during pregnancy. The biological sample may conveniently be a blood sample, or alternatively, could be derived from another tissue, such as placental tissue, provided the tissue is suspected of containing material in which the presence or absence of the polymorphism ΔA can be detected.

The presence of polymorphism ΔA can be detected using methodologies known to those of skill in the art. For example, an assay that may be conducted either at the point of care or in a remotely located laboratory can be used. The method may involve hybridization of a sequence in the biological sample to any of SEQ ID NO:3, SEQ ID NO: 6, a modified sequence based thereon, or a sequence complementary thereto.

By “modified sequence” it is meant a sequence based on one of SEQ ID NO:3 or SEQ ID NO: 6 that comprises a substitution of one or more bases, a modification of one or more bases, a deletion of one or more bases, or a combination of these, that has no material effect on hybridization of the sequence to a sequence in the biological sample bearing the polymorphism ΔA. Such a modified sequence can be modified only to the extent that a person of skill in the art can derive a relatively specific conclusion about the presence or absence of the polymorphism ΔA, comparable to the result that may be derived if the unmodified sequence was used.

A method of detecting a propensity toward or risk of developing preeclampsia is described which comprising assessing a female subject for ΔA/ΔG or ΔA/ΔA genotype at position +1754 in HLA-G exon 8, containing HLA-G mRNA 3′UTR sequence.

There is also described herein the use of a sequence according to SEQ ID NO:3, SEQ ID NO: 6, a modified sequence based thereon, or a sequence complementary thereto, for detection of a polymorphism ΔA at position +1754 in HLA-G mRNA 3′UTR in a biological sample from a female, indicative of a propensity toward preeclampsia, or a risk of development of preeclampsia.

A kit for detection of preeclampsia, a propensity toward preeclampsia, or risk of developing preeclampsia is described. The kit comprises a probe for detecting polymorphism ΔA at position +1754 in HLA-G exon 8 in a biological sample from a female, together with directions for use. The probe may be any probe capable of detecting polymorphism ΔA in the sample, which encompasses a variety of probe types as would be known to those of skill in the art. For example, the probe may comprise SEQ ID NO:3, SEQ SEQ ID NO: 6, a modified sequence based thereon, or a sequence complementary thereto. The kit may be in the form of a chip or biological sensor, a laboratory-based assay, PCR, ELISA, or other known methods, and may be offered as a point of care test or a test that is conducted under laboratory conditions.

The subject may be heterozygous or homozygous, as each option can be considered representative of such a propensity or risk.

The mutation “A” at position 1754 may be referred to herein interchangeably as simply as “the mutation at 1754”, as “1754 ΔA” or as polymorphism ΔA.

### EXPERIMENTAL

### A Single Base-Pair Mutation in the 3′-Untranslated Region (3′-UTR) of HLA-G mRNA Associated with Preeclampsia Influences mRNA Stability In Vitro

This study was initiated to determine whether a DNA polymorphism exists in the HLA-G mRNA 3′UTR, at or near the AUUUA motif, and test whether this polymorphism is associated with preeclampsia (PE) and/or HLA-G mRNA stability.

Materials And Methods

Patients. After obtaining appropriate consent, 29 preeclampsic patients and 15 normal control women were recruited for this study. All the subjects were seen in the labor and delivery suite of the Women's College Hospital (WCH), University of Toronto, Toronto, Canada from 1996 to 1997. Preeclampsia was diagnosed, and sub-classified as mild vs. severe, according to the guideline published by the American College of Obstetricians and Gynecologists (20). Patients who had a multiple gestation or chorioamnionitis were excluded from the study. According to the protocol, the next patient who delivered, having had an uncomplicated pregnancy and delivery was approached to participate. A total of 14 out of 29 of these patients agreed to participate.

Table 1 shows a summary of the clinical characteristics of the study groups. This study was approved and monitored by the ethics committee of WCH.

TABLE 1
Clinical Characteristics of Patients

| | Preeclampsia cases | Normal Controls |
|---|---|---|
| Characteristics | (n = 29) | (n = 15) |
| Age (yrs) | 30.3 ± 4.8 | 32.1 ± 5.3 |
| | (range 24-40) | (range 25-41) |
| Primiparas* | 16/20 (80%) | 9/14 (64%) |
| Gestational Age (weeks)* | 35.3 ± 2.4 | 39.4 ± 1.8 |
| | (range 31-39) | (range 38-42) |
| Birth weight (grams)* | 2319 ± 708 | 3324 ± 482 |
| | (range 1215-3595) | (range 2580-4264) |
| IUGR* | 5/20 (25%) | 0/14 (0%) |
| Disease Severity | mild - 50% | n/a |
| | severe - 50% | |

*p < .05

DNA Extraction and HLA-G Gene Sequence. DNA was extracted from placenta tissues and blood cells by using a phenol/chloroform protocol (21). DNA concentration and purity were measured by UV spectrophotometry at 260/280 nm. The dried DNA pellets were dissolved in 10-20 μl TE buffer, PH 8.0. HLA-G exon 8, containing the HLA-G mRNA 3′UTR sequence, was amplified by polymerase chain reaction (PCR): 200 ng of DNA was made up to a final volume of 50 μl with the following primers: sense: 5′-TGTGGGACTGAGTGGCAAGT-3′ (SEQ ID NO: 7) and anti-sense: 5′TTTGTCTCTAAATTTCAGGAATC-3′ (SEQ ID NO: 8) at initial denaturation of 94° C. for 5 min, 30 cycles of 94° C. for 1 min, 51° C. for 1 min s and 72° C. for 2 min and the final extension step at 72° C. for 10 min.

Each sample PCR product was purified from a 2% low melting point agarose gel by using QIAquick™ Gel-Extraction Kit (Qiagen, Hilden Germany). Approximately 50 ng of the purified products were then sequenced in both directions by using ABI PRIME Big-Dye Terminator Cycle Sequencing Ready Reaction Kit (Applied Biosystems, CA, USA) on an ABI DNA analyzer (Applied Biosystems).

In Vitro Mutagenesis. To generate in vitro mutagenesis during reverse transcription, total RNA from cultured JEG-3 cells was extracted using Trizon™ Reagents (Gibco, Burlington, ON, Canada) according to the manufacturer's manual. Reverse transcription reactions were performed using MMLV reverse transcriptase (Epicentre Technologies, Madison, Wis., USA) with HLA-G gene specific primers HLAGΔG normal sequence: 5′-TAAACTTTTTCATTTAAATG-3′ (SEQ ID NO: 9) and HLAGΔA mutation sequence: 5′-TAAACTTTTTCATTTAAATA-3′ (SEQ ID NO: 3).

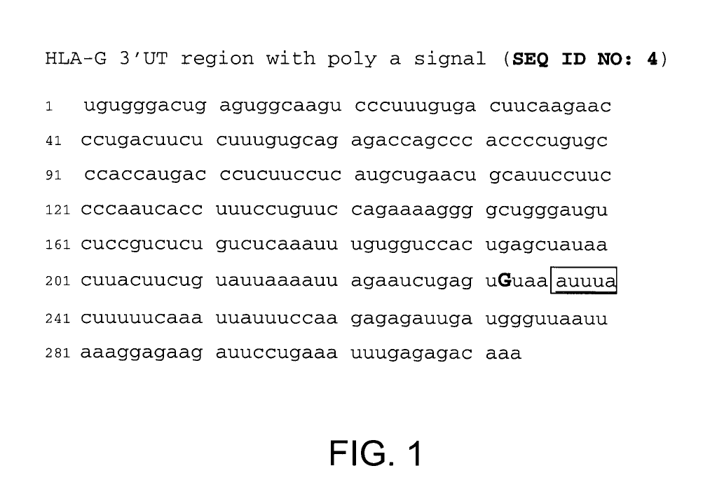

FIG. 1 shows the sequence of HLA-G mRNA 3′ untranslated region. Initial sequences analysis showed a correlation with a mutation in the 3′ untranslated region adjacent to an AUUUA motif.

The primers above (SEQ ID NO: 9 and SEQ ID NO: 3) correspond to HLA-G mRNA 3′UTR nucleotides +1754 to +1773. An excerpted portion of the 3′UT region is represented in FIG. 1 and SEQ ID NO: 4 (see residues 232 to 251) having one base pair different from each other at residue 232 (corresponding to Δt - - - Δc at +1754). The cDNAs were amplified by PCR with the following primers: GF: 5′-CACCACCCTGTCTTTGACTA-3′ (SEQ ID NO: 10) and GB: 3′-ATCTTGGAACAGGGTGGTCC-5′ (SEQ ID NO: 11), denatured at 94° C. for 5 min, and then 35 cycles at 94° C. for 1 min, at 50° C. for 1 min and at 72° C. for 2 min. When stated 5′-3″, the primer noted as SEQ ID NO: 11 is: 5′-CCTGGTGGGACAAGGTTCTA-3′ (SEQ ID NO: 12) The PCR product was checked on a 1.2% argarose gel stained with EB and cloned into a pcDNA™ 3.1 directional TOPO expression vector (Invitrogen Corporation Carlsbad, Calif. USA). Cloned plasmids were transfected into SP/2 myeloma cell line which don't express HLA-G by using Lipofectamine 2000 (Invitrogen) according to the manufacturer's manual.

Transfected SP/02 cell line was cultured in RPMI 1640 supplemented with 10% fetal calf serum in the presence of 50 IU/ml penicillin, 50 mg/ml streptomycin and amphotericin B 50 mg/ml B at 37° C., 5% CO2 for 48 hours.

Actimycin D Study. During the experiments, the cells were washed three times with serum free medium (RPMI1640). The cells were maintained in the serum free medium and actinomycin D (Sigma) was added to the cell culture for 0, 0.5, 1, 2 and 4 hours at a final concentration of 5 μg/ml. The cells were then collected by centrifugation at 4° C., 800 rpm for 5 min and cell pellets were stored at −80° C. until assay. Empty plasmids of pcDNA™ 3.1 directional TOPO expression vectors were also used to detect nonspecific background.

RNA Extraction. Total RNA of the cell pellets at each time points was extracted with Trizon reagents as described above. HLA-G stability was measured by either a RT-PCR-ELISA or a RNAase protection assay as follows:

1) RT-PCR-ELISA. An HLA-G fragment prepared from JEG-3 cells by RT-PCR using primers G256 and G1225 (22), corresponding to nucleotides +256 to +1225, was used as a probe. 1 mg/ml of the fragment was denatured at 95° C. for 5 min, put on ice for 3 min and coated on a 96-well microtiter plate (Dynatec, Chantilly, Va., USA) in 50 ml per well of 0.1 M PBS/1 M NaCl coating buffer (pH=7.2) at 4° C. overnight. Then, the plate was washed twice, dried and stored at −20° C. until use.

The total RNA of each time point was amplified by RT-PCR during 30 cycles (94° C. 1′ 58° C. 1′ 72° C. 2′) in the presence of biotin labeled primer sets (G256 and G1225). 5 ml of each PCR product in triplicate was denatured using 1N NaOH and hybridized to plate coated probe at 50° C. for 2 hours. 50 ml per well of 1:1000 streptoavidin-HRP conjugate (Sigma) diluted in sample buffer (0.01 M PBS/150 mM NaCl, 0.5% block solution, 5 mM EDTA and 1% Tween-20™) was added following 4 washes in this buffer. After one hour incubation at room temperature, the plate was washed 4 times again with sample buffer, and 100 ml of TMB (Sigma) was added. After a 15 min of incubation, color reactions were stopped by adding 50 ml per well of 1 M HCl and read at 450/630 nm at a microplate reader (Awareness Technology Inc. Palm, Fla., USA). The cloned plasmids were used as standard amplified together with sample cDNAs. HLA-G mRNA levels at each time point were determined by comparison to standard.

2) RNase Protection Analysis. RNAase protection experiments were performed by using SuperSignal® RPAIII™ kit (AmBion Inc, Austin, Tex. USA) according to the manufacturer's manual. The HLA-G cDNA probe was prepared from the plasmid by in vitro transcription using T7 RNA polymerase and biotin-UTP. The probe was hybridized to total RNA and treated with RNAase at 37° C. for 2 hours. The protected fragments of HLA-G mRNA were determined by 6% TBE gel electrophoresis and autoradiography. In all experiments, β-actin mRNA was used for control purposes.

Statistical Analysis. HLA-G genotype frequencies were compared to Hardy-Weinberg expectations using χ²-tests. The frequency of the 1754ΔA allele was compared between preeclampsic patients and controls as well as sever and mild subgroups of preeclampsia with a χ²-test. Comparisons of HLA-G mRNA half-life between normal controls and the mutation were carried out by using a Student's t test.

Results

Definition of Detected HLA-G Alleles and Comparison of the Allele Frequencies Between PET Patients and Healthy Controls

We identified a polymorphism, ΔA (mutant) or ΔG (native), located at +1754 of HLA-G gene exon 8 in the HLA-G mRNA 3′UTR that is adjacent to the solitary AUUUA motif in this region. This polymorphism is evident when we studied previously reported HLA-G sequences (23,24).

By DNA sequence analysis, the following placental HLA-G alleles with respect to this polymorphism were observed in the preeclampsia group: ΔG/ΔG=2 ΔG/ΔA=11 and ΔA/ΔA=16, while in normal pregnant women: ΔG/ΔG=11, ΔG/ΔA=0 and ΔA/ΔA=4; X²=15.6; df.=1; P<0.0001. Thus, a greater number of 1754ΔA alleles was found in placenta of women with preeclampsia compared to healthy pregnancy women (Table 2). Furthermore, 10/15 placentas were found to be homozygous ΔA/ΔA in patients with severe PE while only 3/14 was found among those with mild disease (Table 3). Statistical analysis indicated that the homozygous ΔA/ΔA allele genotype was significantly higher in association with severe disease than mild (X²=9.19; df.=1; P=0.00256).

Table 2 illustrates that a greater number of 3952ΔA for the exon 8 polymorphism was found in placenta of women with preeclampsia compared to healthy pregnancy women.

TABLE 2
The number of samples with each genotype for the ΔA allele

| Samples | N | +/+ | ΔA/+ | ΔA/ΔA | ΔA frequency | P value |
|---|---|---|---|---|---|---|
| Control | 15 | 11 | 0 | 4 | 0.071 | |
| Preeclampsia | 29 | 2 | 11 | 16 | 0.549 | <0.0001 |

+. Normal allele,
Δ null allele samples were compared to control by a χ2 test.

Table 3 illustrates that 10/14 placentas were found to be homozygous ΔA/AA in patients with severe PE while only 3/15 were found among those with mild disease. Statistical analysis indicated that the homozygous ΔA/ΔA allele genotype was significantly higher in association with severe disease than mild (χ²=9.19; df.=1; P=0.00256).

TABLE 3
The number of samples with the genotype between mild and
severe preeclampsia for homozygous 1754ΔA allele

| | | | | | 1754ΔA | |
|---|---|---|---|---|---|---|
| Samples | N | +/+ | ΔA/+ | ΔA/ΔA | frequency | P value |
| Severe | 14 | 2 | 2 | 10 | 0.617 | |
| Mild | 15 | 0 | 11 | 3 | 0.321 | 0.0256 |

Effect of the Mutation on the Stability of HLA-G mRNA

Since steady-state mRNA levels can be reduced by either inhibiting transcription or by decreasing mRNA stability and the null allele is adjacent to an AUUUA (SEQ ID NO: 1) motif in the HLA-G mRNA 3′UTR, we deduced that reduced HLA-G protein levels observed in preeclampsia (6-8) may be caused by an increased rate of HLA-G mRNA degradation. To evaluate the HLA-G mRNA stability in association with these alleles, HLA-G mRNA levels was measured at timed intervals after addition of Actinomycin D by two methods: 1) an RT-PCR-ELISA and 2) an RNAase protection assay as described in the Materials and Methods.

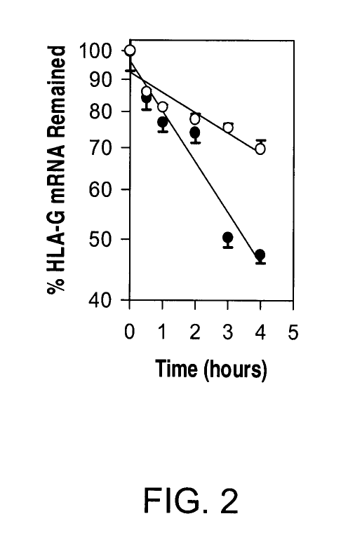

FIG. 2 to FIG. 5 illustrate the results of a variety of methods for evaluation of HLA-G mRNA stability. Transfected SP/02 cells were treated with actinomycin D for various time periods. Solid circles represent mutation (1754 ΔA), while open circles represent control (1754 ΔGA).

FIG. 2 shows HLA-G mRNA expression determined by RT-PCR-ELISA. These data show that levels of HLA-G mRNA with the ΔA allele had decreased to 49.7±1.69% of baseline (mean±SE, N=7) by 3 hours after addition of actinomycin D, whereas the ΔG containing HLA-G mRNA only decreased to 75.4±1.14% (mean±SE, N=7) of baseline, as determined by the RT-PCR-ELISA.

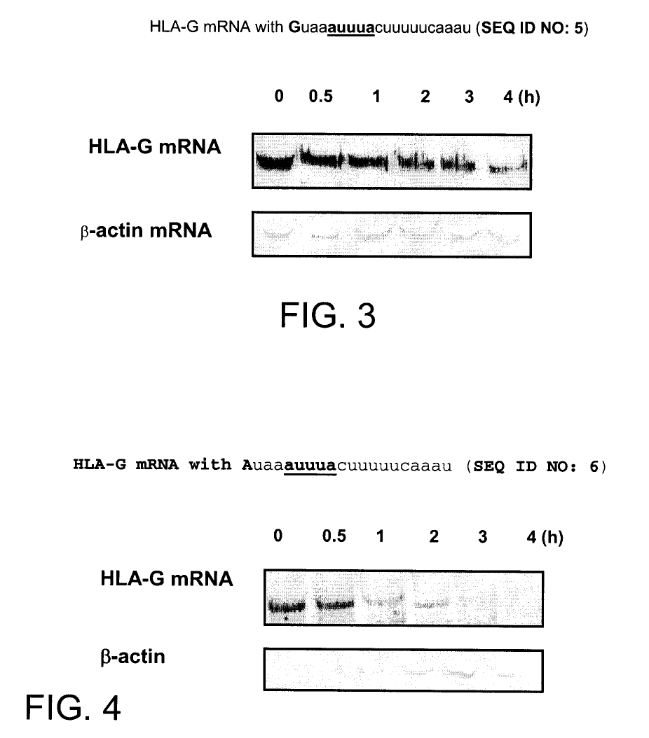

FIG. 3 and FIG. 4 show a comparison of half-lives between HLA-G mRNA with ΔA allele and with ΔG, respectively, by RNAase protection assay. HLA-G mRNA levels in cells translated with the ΔA HLA-G plasmid decayed more rapidly compared to that with ΔG sequence.

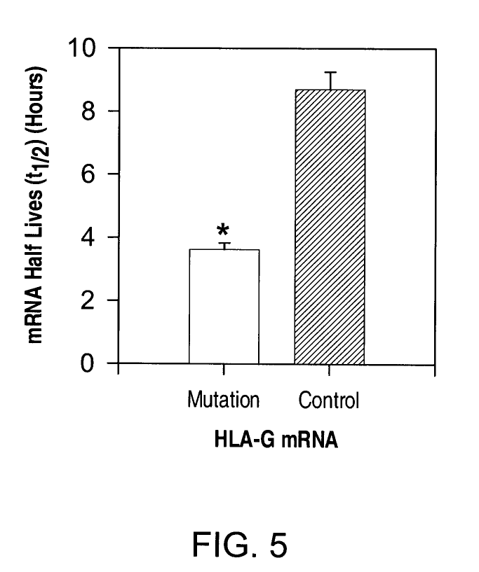

FIG. 5 shows a comparison of half lives between HLA-G mRNA with mutation and control sequences. Statistical analyses showed that the HLA-G mRNA half life in ΔA allele translated cells was significantly shorter than that of the ΔG sequence (3.63±0.203 vs 8.70±0.550 hours, p=0.0001).

Discussion

The results of this study indicate that frequency of the ΔA Allele (1754ΔA) in HLA-G mRNA 3′UTR is significantly higher in placental tissues samples from patients with preeclampsia than that of healthy controls (0.548 vs 0.071, p<0.0001; Table 2). The results also indicate that homozygosity for the ΔA allele is significantly associated with severity of the disease (0.617 vs 0.321, p=0.0256, Table 3).

For reference purposes, an HLA-G 3′ untranslated region of HLA-G exon 8 is provided in SEQ ID NO: 13, showing bases 1 to 1840, and the mutant ΔA at position 1754. The sequence of exon 8 has been described, for example in Zemmour et al., Hum. Immunol. 31 (3), 195-206 (1991) (36).

Since preeclampsia appears to be associated with a poor placentation, it has long been considered that this disease may be a form of maternal immune rejection of the genetically foreign fetus (3). However, cytotrophoblasts don't express the highly immunogenic transplantation antigens, HLA-A, -B and D (25). In fact, these invasive cytotrophobasts that infiltrate maternal tissues during placentation express a unique combination of HLAs, namely HLA-C, -E and -G) (25). Of these, only HLA-C is highly polymorphic. On the other hand, in the mother's the decidua, there are many maternal lymphocytes namely NK cells with various phenotypes. It has been proposed that when a woman is homozygous for B group of NK cells while the fetus is homozygous for the HLA-C2, PE may be more prevalent (26) in these patients. However, the proposal has not been confirmed in vitro (3).

Unlike HLA-C, HLA-G shows a limited polymorphism (27) and a restricted tissue distribution (4). However, a large number of studies have demonstrated that HLA-G plays an important role in maternal-fetal immunotolerance by inhibiting activation of maternal T and NK cells resident in the deciduas (28). So, taking the biology of HLA-G into consideration, the poor placentation might be occurring as a result of lower expression of HLA-G by invasive trophoblasts. It has been shown that both HLA-G gene transcription (6-8) and translation (7-11) are reduced in women with preeclampsia.

The steady-state levels of a particular mRNA depend not only upon its synthesis but also on its rate of degradation. To explore underlying mechanisms for the reduced HLA-G gene expression, some studies have been performed to identify any HLA-G polymorphism in the pathophysiology of PE. However, most polymorphisms discovered in exons 2 and 3(29-33), expect a silent CAC-CAT at codon 93 and a 14 bp in the 3′UTR, have no significant association with PE (30, 34). Furthermore, as the two significant polymorphisms are silent, it is difficult to determine the relevance of this association (5) and effect of the polymorphisms on HLA-G gene expression was not confirmed in vitro.

In this example, the null allele was discovered in the HLA-G mRNA 3′UTR adjacent to a AUUUA motif (SEQ ID NO: 1), suggesting it may have an effect on HLA-G gene transcription. In order to demonstrate the hypothesis, we carried out an in vitro mutagenesis. The results of study demonstrated that the null allele has significant effect on HLA-G mRNA stability. This may help explain how HLA-G transcription levels are reduced in PE (FIG. 2).

The large number of epidemiologic studies carried out on PE indicate that it can be considered a heritable disorder (5). In one of the largest epidemiological studies, it was suggested both mother and the fetus contribute to the risk of PE, with the contribution of the fetus being affected by paternal genes (35). In this study, the number of the samples would not have sufficient power to confirm that the ΔA allele is present in all populations, and is a unique mechanism for the reduced HLA-G expression in PE.

In summary, according to the invention it has been shown that the presence of a single base pair point mutation in the HLA-G gene 3′UT region appears to be associated with PE and with decreased RNA stability in vitro. Therefore, the presence of this ΔA mutation may be an important predisposing factor to account for some subjects exhibiting lower placental HLA-G expression in association with PE.

The above-described embodiments of the present invention are intended to be examples only. Alterations, modifications and variations may be effected to the particular embodiments by those of skill in the art without departing from the scope of the invention, which is defined solely by the claims appended hereto.

### REFERENCES

1. Sibai B, Dekker G, Kupferminc M. Pre-eclampsia. Lancet 2005:365-99.
2. Widschwendter M, Schrocksnadel H, Mortl M G. Preeclampsia: a disorder of placental mitochondria? Mol Med Today 1998; 4:286-91
3. Redman C W, Sargent I L. Latest advances in understanding preeclampsia. Science 2005, 308:1592-4.
4. McMaster M T, Librach C L, Zhou Y, Lim K H, Janatpour M J, DeMars R, Kovats S, Dansky C, Fisher S J: Human placenta HLA-G expression is restricted to differentiated cytotrophoblasts. J Immunol 1995; 154: 3771-8.
5. O'Brren M, Dausset J, Carosella E D, Morean P: Analysis of the role of HLA-G in preeclampsia. Human Immunol 2000; 61:1126-31.
6. Colbern G T, Chiang M H, Main E K. Expression of the non-classic histocompatibility antigen HLA-G by preeclamptic placenta. Am J Obstet Gynecol. 1994; 170:1244-50
7. Hara N, Fujii T, Yamashita T, Kozuna S, Okai T, Takotani Y: Altered expression of human leukocyte antigen G (HLA-G) on extravillous trophoblasts in preeclampsia: Immunohistological demonstration with anti-HLA-G specific antibody “87G” and anti-cytokeratin antibody “CAM5.1”. Am J Reprod Immunol 1996; 36:349-58.
8. Lim K H, Zhou Y, Janatpour, McMaster M, Bass K, Chun S H, Fisher S J: human cytotrophoblast differentiation/invasion is abnormal in pre-eclampsia. Am J Phathol 1997; 151:1809-18.
9. Goldman-wohl D S, Greenfield A I: Hochner-celnikier D, Cross J, Fisher S, Yagel S: lack of human leukocyte antigen-G expression in extravillous trophoblast is associated with pre-eclampsia. Mol Human Reprod 2000; 6:88-95.
10. Yie S M, L H Li, Y M Li, C F Librach: serum and placenta HLA-G decreased in women with preeclampsia. Am J Obs Gyn 2004; 191:525-9.
11. Yie S M, R N Tailer, C F Librach: Low Plasma HLA-G protein concentrations in early gestation indicate the development of preeclampsia later in pregnancy Am J Obs Gyn 2005; 193:204-8.
12. Hylenius S, Andersen A M, Melbye M, Hviid T V. Association between HLA-G genotype and risk of pre-eclampsia: a case-control study using family triads. Mol Hum Reprod. 2004; 10:237-46.
13. Aldrich C, Verp M S, Walker M A, Ober C A null mutation in HLA-G is not associated with preeclampsia or intrauterine growth retardation. J Reprod Immunol. 2000; 47:41-8.
14. Humphrey K E, Harrison G A, Cooper D W, Wilton A N, Brennecke S P, Trudinger B J. HLA-G deletion polymorphism and pre-eclampsia/eclampsia. Br J Obstet Gynaecol. 1995; 102:707-10.
15. Caput D, Beutler B, Hartog K, Thayer R, Brown-Shimer S, Cerami A, Identification of a common nucleotide sequence in the 3′-untranslated region of mRNA molecules specifying inflammatory mediators. Proc Natl Acad USA 1986; 83:1670-4.
16. Shaw G, Kamen R. A conserved AU sequence from the 3′ untranslated region of GM-CSF mRNA mediates selective mRNA degradation. Cell 1986; 46:659-67.
17. Good P J. The role of elav-like genes, a conserved family encoding RNA-binding proteins, in growth and development. Semin Cell Dev Biol 1997; 8:577-84.
18. Henics T, Nagy E, Oh H J, Csermely P, von Gabain A, Subjeck J R. Mammalian Hsp70 and Hsp110 proteins bind to RNA motifs involved in mRNA stability. J Biol Chem 1999; 274:17318-24.
19. Zhang W, Wagner B J, Ehrenman K, Schaefer A W, DeMaria C T, Crater D, DeHaven K, Long L, Brewer G. Purification, characterization, and cDNA cloning of an AU-rich element RNA-binding protein, AUF1. Mol Cell Biol 1993; 13:7652-65.
20. ACOG Committee on Obstetric Practice. ACOG practice bulletin. Diagnosis and management of preeclampsia and eclampsia. Number 33, January 2002. American College of Obstetricians and Gynecologists. Int J Gynaecol Obstet. 2002; 77:67-75
21. Bell G I, Karam J H, Rutter W J: Polymorphic DNA region adjacent to the 5′end of human insulin gene. Proc Natl Acad Sci USA 1981: 78:5759-
22. Kirszenbaum M, Moreau P, Gluckman E, Dausset J, Carosella E An alternatively spliced form of HLA-G mRNA in human trophoblasts and evidence for the presence of HLA-G transcript in adult lymphocytes. Proc Natl Acad Sci USA. 1994 May 10; 91(10): 4209-4213.
23. Geraghty, D E., Koller, B H. and Orr, H T. A human major histocompatibility complex class I gene that encodes a protein with a shortened cytoplasmic segment. Proc. Natl. Acad. Sci. U.S.A. 1987; 84:9145-9.
24. NCI-CGAP. National Cancer Institute, Cancer Genome Anatomy Project (CGAP), Tumor Gene Index. Unpublished (1997).
25. Moffett-King A. Natural killer cells and pregnancy Nat Rev. Immunol 2002; 2:656-63.
26. Parham P. MHC class I molecules and KIRs in human history, health and survival. Nat Rev. Immunol 2005; 5:201-14.
27. Le Bouteiller P, Mallet V. HLA-G and pregnancy. Rev. Reprod 1997; 2:7-13.
28. Carosella E D, Moreau P, Le Maoult J, Le Discorde M, Dausset J, Rouas-Freiss N. HLA-G molecules: from maternal-fetal tolerance to tissue acceptance. Adv Immunol 2003; 81:199-252.
29. van der Ven K, Ober C, HLA-G polymorphisms in African American. J Immuonol 1999; 153:5628-33.
30. Kirszenbaum M, Djoulah S, Hors J, Prost S, Dausset J, Carosella E D. Polymorphism of HLA-G gene and protein. J Reprod Immunol 1999; 43:105-9.
31. Suarez M B, Morales P, Castro M J, Fernandez V, Varela P, Alvarez M, Martinez-Laso J, Arnaiz-Villena A. A new HLA-G allele (HLA-G*0105N) and its distribution in the Spanish population. Immunogenetics 1997:45:464-5.
32. Ober C, Aldrich C, Rosinsky B, Robertson A, Walker M A, Willadsen S, Verp M S, Geraghty D E, Hunt J S. HLA-G1 protein expression is not essential for fetal survival. Placenta 1998; 19:127-32.
33. Aldrich C, Verp M S, Walker M A, Ober C. A null mutation in HLA-G is not associated with preeclampsia or intrauterine growth retardation. J Reprod Immunol 2000; 47:41-8.
34. Hvvid T Y. HLA-G genotype is associated with fetoplacental growth. Human Immunol 2004; 65:586-93.
35. Lie R T, Rasmussen S, Brunborg H, Gjessing H K, Lie-Nielsen E, Irgens L M. Fetal and maternal contribution to risk of pre-eclampsia: population based study. Br. Med J 1998; 316:1343-7.
36. Zemmour et al., Hum. Immunol. 31 (3), 195-206 (1991) (35).

1. A method of detecting a propensity toward preeclampsia comprising assessing a biological sample from a female subject for the presence of polymorphism ΔA at position +1754 in an HLA-G mRNA 3′UTR sequence, wherein the presence of polymorphism ΔA indicates a propensity toward preeclampsia.

2. The method of claim 1, wherein said biological sample is obtained from placenta tissue or blood.

3. The method of claim 1 wherein the presence of polymorphism ΔA is detected by hybridization to SEQ ID NO:3, SEQ ID NO: 6, a modified sequence based thereon, or a sequence complementary thereto, wherein the modified sequence comprises a substitution of one or more bases, a modification of one or more bases, a deletion of one or more bases, or a combination of these, that has no material effect on hybridization of the sequence to a sequence in the biological sample bearing the polymorphism ΔA.

4. The method of claim 3 wherein the polymorphism ΔA is detected by hybridization to SEQ ID NO:3, SEQ ID NO: 6 or a sequence complementary thereto.

5. A method of detecting a propensity toward preeclampsia comprising assessing a female subject for ΔA/ΔG or ΔA/ΔA genotype at position +1754 in HLA-G exon 8, containing HLA-G mRNA 3′UTR sequence.

6. Use of a sequence according to SEQ ID NO:3, SEQ ID NO: 6, a modified sequence based thereon, or a sequence complementary thereto, for detection of a polymorphism ΔA at position +1754 in HLA-G mRNA 3′UTR in a biological sample from a female, indicative of a propensity toward preeclampsia; wherein the modified sequence comprises a substitution of one or more bases, a modification of one or more bases, a deletion of one or more bases, or a combination of these, that has no material effect on hybridization of the sequence to a sequence in the biological sample bearing the polymorphism ΔA.

7. The use of claim 6, wherein SEQ ID NO:3, SEQ ID NO: 6 or a sequence complementary thereto is used.

8. A kit for detection of a propensity toward preeclampsia comprising: a probe for detecting polymorphism ΔA at position +1754 in HLA-G exon 8 in a biological sample from a female; and directions for use, wherein said polymorphism ΔA is indicative of a positive propensity.

9. The kit of claim 7, wherein the probe comprises SEQ ID NO:3, SEQ ID NO: 6, a modified sequence based thereon, or a sequence complementary thereto, wherein the modified sequence comprises a substitution of one or more bases, a modification of one or more bases, a deletion of one or more bases, or a combination of these, that has no material effect on hybridization of the sequence to a sequence in the biological sample bearing the polymorphism ΔA.

10. The kit of claim 9, wherein the probe comprises SEQ ID NO:3, SEQ ID NO: 6 or a sequence complementary thereto.


SEQUENCE LISTING (13 sequences)

SEQ ID NO: 1 - RNA, 5 nt, Homo sapiens
auuua

SEQ ID NO: 2 - RNA, 8 nt, Homo sapiens
auuauuuu

SEQ ID NO: 3 - DNA, 20 bp, Homo sapiens
taaactttttcatttaaata

SEQ ID NO: 4 - RNA, 313 nt, Homo sapiens
ugugggacugaguggcaagucccuuugugacuucaagaacccugacuucucuuugugcagagaccagcccaccccugugcccaccaugacccucuuccucaugcugaacugcauuccuuccccaaucaccuuuccuguuccagaaaaggggcugggaugucuccgucucugucucaaauuugugguccacugagcuauaacuuacuucuguauuaaaauuagaaucugaguguaaauuuacuuuuucaaauuauuuccaagagagauugauggguuaauuaaaggagaagauuccugaaauuugagagacaaa

SEQ ID NO: 5 - RNA, 20 nt, Homo sapiens
guaaauuuacuuuuucaaau

SEQ ID NO: 6 - RNA, 20 nt, Homo sapiens
auaaauuuacuuuuucaaau

SEQ ID NO: 7 - DNA, 20 bp, Artificial
  sense primer
tgtgggactgagtggcaagt

SEQ ID NO: 8 - DNA, 23 bp, Artificial
  anti-sense primer
tttgtctctaaatttcaggaatc

SEQ ID NO: 9 - DNA, 20 bp, Homo sapiens
taaactttttcatttaaatg

SEQ ID NO: 10 - DNA, 20 bp, Artificial
  primer
caccaccctgtctttgacta

SEQ ID NO: 11 - DNA, 20 bp, Artificial
  primer
atcttggaacagggtggtcc

SEQ ID NO: 12 - DNA, 20 bp, Artificial
  primer
cctggtgggacaaggttcta

SEQ ID NO: 13 - DNA, 1840 bp, Homo sapiens
cccattaggtgacaggtttttagagaagccaatcacgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaagtaagtatgaaggaggctgatccctgagatccttgggatcttgtgtttgggagccatgggggagctcacccaccccacaattcctcctctggccacatctcctgtggtctctgaccaggtgctgtttttgttctactctaggcagtgacagtgcccagggctctaatgtgtctctcacggcttgtaaatgtgacaccccggggggcctgatgtgtgtgggttgttgaggggaacaggggacatagctgtgctatgaggtttctttgacttcaatgtattgagcatgtgatgggctgtttaaagtgtcacccctcactgtgactgatatgaatttgttcatgaatatttttctgtagtgtgaaacagctgccctgtgtgggactgagtggcaagtccctttgtgacttcaagaaccctgacttctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaatttactttttcaaattatttccaagagagattgatgggttaattaaaggagaagattcctgaaatttgagagacaaaataaa